(19) United States
(12) Patent Application Publication
(10) Pub. No.: US 2026/0060781 A1
(43) Pub. Date: Mar. 5, 2026

(54) Process For The Production Of Ceramic And Glass-Ceramic Dental Restorations

(71) Applicant: Ivoclar Vivadent AG, Schaan (LI)

(72) Inventors: Nadja Cavegn, Planken (LI); Lorenz Josef Bonderer, Sargans (CH)

(21) Appl. No.: 19/311,625

(22) Filed: Aug. 27, 2025

(30) Foreign Application Priority Data
Aug. 30, 2024 (EP) 24197511.9

(51) Int. Cl. A61C 5/77; A61K 6/833

(52) U.S. Cl. CPC A61C 5/77; A61K 6/833

(57) ABSTRACT

Method for producing an all-ceramic dental restoration with a one-piece core and a shell structure, in which a digital construction model of the dental restoration is constructed, the CAD data set obtained is then divided into at least two separate CAD partial data sets. One partial data set defines the contour of the core structure and a second partial data set defines the contour of the shell structure. A green body of the core structure is produced by the first CAD partial data set and a green body of the shell structure of the restoration is produced by the second CAD partial data set. The core and shell structure are then joined together in the green state. The green body is then subjected to heat treatment to remove the binder and the component is then sintered to obtain the finished dental restoration.

### CROSS-REFERENCE TO RELATED APPLICATIONS

This application claims priority to European patent application No. 24197511.9 filed on Aug. 30, 2024, the disclosure of which is incorporated herein by reference in its entirety.

### TECHNICAL FIELD

The present invention relates to a method for producing ceramic and glass-ceramic dental restorations, such as dental inlays, onlays, veneers, crowns, bridges and frameworks.

### BACKGROUND

For esthetic reasons, dental restorations are increasingly being made entirely of ceramic. In practice, a framework made of oxide ceramic is often veneered with glass ceramic.

DE 10 2005 042 091 A1 discloses a method for manufacturing ceramic dental prostheses in which a framework is milled from an oxide ceramic blank, for example from aluminum oxide or zirconium dioxide, and a veneer is milled from a glass ceramic block, for example from silicate ceramic. The framework and veneer are produced using the CAD/CAM process. The framework and veneer are then joined and fired using a low-viscosity, low-fusing ceramic material. After sintering, the occlusion is milled and the restoration is completed in a glaze firing.

WO 2007/051447 A1 and corresponding US2009233258 A1, which US published application is hereby incorporated by reference, disclose a method for producing multilayer dental prostheses, in which a supporting framework structure is first produced from metallic or ceramic materials or polymers by means of CAD/CAM and then a functional layer serving as a veneer is applied. The functional layer is preferably produced using additive processes such as hot pressing, die casting or slip casting.

The production of ceramic molded parts by milling is disadvantageous because ceramic materials are very hard and the processing is therefore very time-consuming and associated with high wear of the grinding devices and milling cutters used. To avoid these disadvantages, WO 2010/010087 A1 and corresponding U.S. Pat. No. 10,322,973 B2, which US patent is hereby incorporated by reference, propose the use of porous ceramic bodies for the manufacture of veneers, which are applied to a metal or ceramic framework. The veneers can consist of several layers whose interface corresponds to the dentin/enamel interface of natural or artificial teeth. They are produced by pressing commercially available veneering ceramic powders into blocks and then pre-sintering them. A CAD/CAM system is used to mill veneers from the blocks, which are then fired onto zirconia copings and densely sintered. The different shrinkage behavior of the framework and veneering material must be taken into account in this procedure.

DE 10 2010 002 484 A1 and corresponding U.S. Pat. No. 8,775,131 B2, which US patent is hereby incorporated by reference, disclose the manufacture of dental crowns with a core of oxide ceramic and an outer layer of plastic, oxide ceramic or glass ceramic. The interface between the core and the outer layer follows the dentin-enamel boundary of a natural tooth. In this way, an appearance similar to that of a natural tooth is said to be achieved. The core is produced using computer-aided processes, such as CAD/CAM or generative processes, while the outer layer is produced by manual layering, overpressing or using computer-aided processes. The outer layer is bonded to the core by gluing, sintering or polymerization.

DE 10 2016 115 916 A1 discloses a method for generating processing data for the production of dental prostheses with a ceramic veneer and a carrier. The method takes into account different shrinkage factors for the support and the veneer and is said to enable veneers and supports made of different materials to be sintered together for the first time. The problem is that the support and veneer shell only fully adapt to each other during final sintering, which makes it difficult to position the shell precisely on the support before sintering and increases the risk of errors.

In addition to traditional milling processes, generative processes such as stereolithography are increasingly being used to manufacture dental restorations. In stereolithography, a molded part is built up layer by layer from a liquid and hardenable monomer resin using computer-aided design data (CAD data) (A. Beil, Fertigung von Mikro-Bauteilen mittels Stereolithographie [Production of micro-components using stereolithography], Düsseldorf 2002, VDI-Publisher 3 ff.). Stereolithographic processes are advantageous in the production of dental moldings made of ceramic materials because they enable a significant simplification of the manual molding and casting processes as well as the milling and grinding operations and avoid the large loss of material that occurs with non-generative processes. Since a complete digital process chain is now established, the classic process steps for the production of dental restorations can be replaced by the digitalization of the model, the virtual design of the dental mold and its generative stereolithographic production.

For the stereolithographic production of ceramic molded parts, a ceramic green body is first produced by layer-by-layer radiation hardening of a ceramic slip, which is then sintered into a dense ceramic molded body after debinding. The green body is also called a green part. Debinding refers to the expulsion of the binder. Herein, the binder used is usually broken down and removed by heating the green body to a temperature of approx. 80° C. to 600° C. through thermal and thermo-chemical processes to form volatile components. It is essential that the formation of cracks and deformations is avoided as far as possible.

Debinding is a critical step in the process. There is a high risk that the component will be damaged by the gases produced during the decomposition of the organic matrix and the pressure exerted by them. Even small defects between the individual construction layers can lead to cracks or even complete destruction of the component during debinding. This risk can be reduced by increasing the debinding time, but this greatly increases the process time.

The debinded component is sintered during high-temperature firing in a sintering furnace. The finely dispersed ceramic powder is compacted and solidified by the effect of temperature below the melting temperature of the main components, making the porous component smaller and increasing its strength.

U.S. Pat. No. 5,496,682, which is hereby incorporated by reference, discloses light-curable compositions for the production of three-dimensional bodies by stereolithography which contain 40 to 70% by volume of ceramic or metal particles, 10 to 35% by weight of monomer, 1 to 10% by weight of photoinitiator, 1 to 10% by weight of dispersant and preferably also solvent, plasticizer and coupling agent.

U.S. Pat. No. 6,117,612, which is hereby incorporated by reference, describes resins for the stereolithographic production of sintered ceramic or metal parts. The resins have a viscosity of less than 3000 mPa·s. Monomers with low viscosity are used for their production, preferably in aqueous solution. The use of dispersants is intended to achieve a high solids content with low viscosity.

DE 10 2005 058 116 A1 discloses suspensions for the stereolithographic production of ceramic implants in the manner described in U.S. Pat. No. 6,117,612, which do not contain any diluents such as water or organic solvents. The viscosity of the suspension is adjusted to less than 20 Pa·s by varying the concentration of a dispersant. Alkylammonium salts of copolymers with acidic groups are used as the dispersant, whereby these can also be layered onto the particles of the ceramic powder.

US 2005/0090575 A1, which is hereby incorporated by reference, describes methods and compositions for the stereolithographic production of ceramic components. It is stated that molded parts produced with the liquid materials known from U.S. Pat. No. 5,496,682 are soft and therefore require an additional hardening step in order to avoid deformation during firing, while molded parts obtained from paste-like materials build up internal stresses during debinding, which lead to cracks during sintering. To avoid these problems, plasticizers are used and the amount of ceramic powder is selected so that the viscosity of the compositions is at least 10,000 Pa·s.

EP 2 233 449 A1 and corresponding U.S. Pat. No. 8,133,831 B2, which US patent is hereby incorporated by reference, disclose slurries for the production of ceramic molded parts using hot-melt inkjet printing processes, which contain ceramic particles, wax and at least one radically polymerizable wax, which produce green bodies that can be debinded without cracking. The disadvantage of these slurries is that they tend to segregate in the liquid state when left to stand for a long time. This is not critical in hot melt inkjet processes because the slurries are only present in liquid form during the printing process, i.e. for a relatively short period of time. In stereolithographic processes, however, the slurries must be stable in liquid form for longer periods of time, which means in particular hat the particles dispersed in the slurry must not settle prematurely.

### SUMMARY

It is an object of the present invention to provide methods and materials for the manufacture of all-ceramic dental restorations with high strength, which do not have the disadvantages of the prior art and which enable the simplified manufacture of esthetically sophisticated restorations with a natural appearance.

### BRIEF DESCRIPTION OF THE DRAWINGS

In the drawings:

FIG. 1 shows a computer-designed core structure of an anterior crown with mamelons in front view.

FIG. 2 shows a rear view of the shell structure matching the core structure shown in FIG. 1.

FIG. 3 shows the printed, debinded and separately sintered shell structure (left) and core structure (right). The shell and core structure were sintered separately to show color differences more clearly.

FIG. 4 shows another view of the shell (left) and core (right) structure shown in FIG. 3.

FIG. 5 shows an anterior crown composed of separately printed core (dentin) and shell (incisal) structures. The printed parts were joined in the green state and debinded and sintered together.

FIG. 6 shows the anterior crown shown in FIG. 5 after polishing and glazing.

### DETAILED DESCRIPTION

According to the invention, a process is provided in which dental restorations are produced with a one-piece framework structure and a matching veneer structure by producing the framework structure and the veneer structure separately from one another using an additive process and then joining them together to form the dental restoration. The framework structure is also referred to here as the core structure or core. The veneering structure can be in one piece or comprise several components, which together form the veneering structure. The veneering structure and its components are also referred to as a shell structure or shell. If the veneering structure comprises several components, these are preferably also manufactured separately from one another.

In the first step of the process according to the invention, a CAD (computer-aided design) program is used to construct a digital design model of the dental restoration in accordance with the anatomical and clinical requirements. The CAD data set of the restoration obtained in this way is then divided into at least two separate but not mutually independent partial data sets (file splitting), with a first CAD partial data set defining the contours of the inner core structure (core) and a second CAD partial data set defining the contours of the outer shell structure (shell) of the dental restoration. The second partial data set can in turn be subdivided into two or more partial data sets, each comprising a part of the contour of the outer shell structure. One or more joining gaps can be provided at defined points between the contours. As a result, at least two CAD data sets are obtained, each of which is saved by the CAD software in a suitable file format, preferably in STL format. The CAD data sets are then used to generate processing data for additive manufacturing of the core and shell structure(s) using CAM (computer-aided manufacturing) software. The final output format is at least two separate output files. The core and shell structure(s) are then additively manufactured independently of each other in a computer-controlled process using the generated data sets, preferably by stereolithography.

Core and shell structure(s) are designed according to the desired requirements. The core structure is usually more intensely colored and less translucent than the less colored but more translucent shell structure. Their respective characteristics are individually adapted to the esthetic requirements of the practitioner or the patient or the conditions of the remaining teeth.

To fabricate the dental restoration, a green body of the core structure of the restoration is preferably produced using the first CAD part data set by curing a first slurry by locally applying radiation energy to form the geometric shape of the core structure.

In a further step, a green body of a shell structure of the restoration is produced using the second CAD part data set by curing a second slurry by locally applying radiation energy to form the geometric shape of the shell structure.

If the shell structure comprises more than one component, a separate green body is created for each part of the shell structure. To produce multi-part shell structures, the second CAD part data set of the digital design model is divided into two or more further CAD data sets (n_{output file}>2), each of which comprises a part of the contour of the shell structure. The components are then manufactured using these CAD shell structure part data sets by hardening a second slip using the first CAD shell structure part data set by locally applying radiation energy to form the geometric shape of a first component of the shell structure, separately, a further slip is cured by means of a second CAD shell structure part data set by local introduction of radiation energy to form the geometric shape of a second component of the shell structure, and this step is repeated as often as necessary according to the number of CAD shell structure part data sets in order to produce the desired number of components. Each repetition produces one green body of a component of the shell structure.

To produce the components of the shell structure, the same or a different slip can be used for each component. Preferably, the components are made from different, particularly preferably from differently colored slurries.

In a subsequent step, the core and shell structure or core structure and the components of the shell structure are joined together in the green state in order to obtain a joined green body of the dental restoration. Preferably, a further slurry is used to improve the bonding of the individual parts, which is referred to here as a joining slip. The joining slip is preferably polymerizable, particularly preferably radically polymerizable. Photoreactive joining slurries are particularly preferred, i.e. slurries that can be cured by irradiation with light, and the polymerization of the joining slip is preferably initiated by exposing the slurry to light. The polymerization of the joining slip enables a firm bond of the joined individual elements of the dental restoration in the green state.

Before the parts are joined and before the joining slip is applied, the joining surfaces of the core and/or shell structure can be individually characterized by the targeted application of intensively colored slurries. Slurries with the same composition as the slurries used to produce the core and shell structure, but with a higher content of color components, are particularly suitable for this purpose. These intensively colored slurries are preferably photoreactive and can be locally fixed by irradiation before the joining slip is applied and the individual parts of the dental restoration are finally joined together.

The green body of the dental restoration is then subjected to heat treatment in a further step to remove the binder (debinding) in order to obtain a brown body of the dental restoration. In a subsequent step, the brown body of the dental restoration is sintered to obtain the finished dental restoration.

The green bodies of the core and shell structure are preferably produced by stereolithography. In this process, ceramic green bodies of the core and shell structure are produced by layer-by-layer radiation curing of ceramic slurries that are flowable at processing temperature, which are then joined together to form the green body of the dental restoration. Joining can be done manually. Preferably, a joining slip is used to join the green bodies. It is particularly preferable to use a photoreactive joining slip and to harden it after joining by irradiating it with light of a suitable wavelength. This increases the strength of the green body of the dental restoration, facilitates its handling and reduces the risk of damage.

The green body of the dental restoration is then debinded, preferably by heating it to a temperature of 90° C. to 600° C.

The resulting brown body of the dental restoration is then sintered to form a dense ceramic molded body. Sintering is preferably carried out by heating to a temperature of 650 to 1800° C., for example in a sintering furnace. The sintering conditions depend on the construction material used. Brown bodies made of glass ceramic are preferably sintered at a temperature of 650 to 1100° C., particularly preferably 700 to 950° C., brown bodies made of zirconium dioxide preferably at 1100 to 1600° C., particularly preferably 1300 to 1500° C., and brown bodies made of aluminum oxide preferably at 1400 to 1800° C., particularly preferably 1600 to 1700° C.

The ceramic moldings produced according to the method of the invention are characterized by high strength and great precision. The flexural strength according to ISO 6872 is preferably above 100 MPa, in particular in the range of 150 to 500 MPa, for molded bodies made of glass ceramic. Molded bodies made of Al₂O₃ have a flexural strength of preferably more than 300 MPa, in particular from 500 to 700 MPa, molded bodies made of ZrO₂ of more than 500 MPa, in particular from 800 to 1100 MPa.

According to the invention, slurries with similar coefficients of thermal expansion and similar sintering shrinkage are used to produce the core and the shell structure and, if applicable, as joining slips, so that the green body of the dental restoration can be debinded and sintered without any problems. The matching of thermal expansion coefficients and sintering shrinkage enables the green bodies of the core and shell structure to be produced with a precise fit, as no different shrinkage behavior needs to be taken into account. This considerably simplifies the joining of core and shell structure and reduces the risk of errors, for example due to unintentional displacement of the shell structure on the core.

To facilitate the joining of the core and shell structure, the core and shell structure can be provided with insertion grooves, joints, ribs or similar structures that ideally align the core and shell with each other and enable a clear, form-fit alignment of the molded parts. Such structures can also take on optical functions after sintering. For example, they can imitate the natural mamelon structure of teeth.

The dental restorations according to the invention comprise a supporting core structure and a shell structure. The shell structure is attached to the core structure. The core structure is attached to the tooth or teeth to be restored or to an implant or implants when the finished restoration is placed in the patient's mouth. The shell structure can completely or preferably partially surround or cover the surface of the core. The shell structure of the dental restoration may comprise one or more, preferably 1 or 2, components which together form the shell structure. Preferably, the shell structure contains only one component.

The use of several components is more complex, but advantageous because core structures with undercuts can be better encapsulated. In addition, splitting the shell structure into several components can be advantageous in terms of production technology. For example, it is easier to produce two separate components to cover the front and back of a core structure than a one-piece shell structure that covers both areas. The risk of stresses and air pockets forming during joining can also be reduced by using multi-part shell structures. The use of multiple components can also have aesthetic advantages. For example, several different colored shell structures can be placed on top of each other to achieve a particularly natural look.

For the production of the core and shell structure and as joining slips, slurries are preferably used that comprise
(a) at least one radically polymerizable monomer,
(b) at least one photoinitiator and
(c) ceramic, glass and/or glass-ceramic particles.

The slurries according to the invention contain as monomer (a) at least one (meth)acrylate and/or (meth)acrylamide, preferably mono- or multifunctional (meth)acrylates, or a mixture thereof. Particularly preferred are materials which contain at least one multifunctional (meth)acrylate or a mixture of mono- and multifunctional (meth)acrylates as radically polymerizable monomer. Monofunctional (meth)acrylates are compounds with one, multifunctional (meth)acrylates are compounds with two or more, preferably 2 to 6, radically polymerizable groups.

Particularly preferred mono- or multifunctional (meth)acrylates are methyl-, ethyl-, 2-hydroxyethyl-, butyl-, benzyl-, tetrahydrofurfuryl- or isobornyl (meth)acrylate, p-cumyl-phenoxyethylene glycol methacrylate (CMP-1E), bisphenol A di(meth)acrylate, bis-G(M)A (an addition product of (meth)acrylic acid and bisphenol A diglycidyl ether), ethoxy- or propoxylated bisphenol A di(meth)acrylate, such as bisphenol-A-di(meth)acrylate with 3 ethoxy groups (SR-348C=methacrylate; SR-349=acrylate, Sartomer) or 2 ethoxy groups (SR-348L=methacrylate, Sartomer), 2,2-bis[4-(2-(meth)acryloxy propoxy) phenyl]propan, UD(M)A (an addition product of 2-hydroxy ethyl(meth)acrylate and 2,2,4- or 2,4,4-trimethyl hexamethylene-1,6-diisocyanate), di-, tri-, tetra-, penta-, hexa- or hepta-ethylene glycol di(meth)acrylat, di-, tri-, tetra-, penta-, hexa- or hepta-propylene glycol di(meth)acrylate, trimethylolpropane tri(meth)acrylate, pentaerythritol tetra(meth)acrylate, as well as glycerol di(meth)acrylate and tri(meth)acrylate, 1,4-butanedioldi(meth)acrylate, 1,10-decanedioldi(meth)acrylate (D₃MA), 1,12-dodecanedioldi(meth)acrylate, oligomeric polyether-, polyester-, epoxy-, urethane-(meth)acrylates, tricyclodecanedimethanol di(meth)acrylate and mixtures thereof.

Particularly preferred are mono-, di-, trifunctional acrylates and methacrylates with a molecular weight of <1000 g/mol, e.g. aliphatic urethane diacrylates, phthalic acid HEA esters (Photomer 4173), pyromellitic acid di-HEA esters (HEA=2-hydroxyethyl acrylate), bisphenol A di(meth)acrylate, bis-G(M)A (an addition product of (meth)acrylic acid and bisphenol A di-glycidyl ether), ethoxy- or propoxylated bisphenol A di(meth)acrylate, such as bisphenol A di(meth)acrylate (SR-348C, SR-349, SR-348L), 2,2-bis[4-(2-(meth)acryloxy propoxy)phenyl]propane, UD(M)A, triethylene glycol di(meth)acrylate (TEGD(M)A), tricyclodecanedimethanol di(meth)acrylate, ethoxylated or propoxylated trimethylolpropane tri(meth) acrylate, e.g. 3-fold propoxylated trimethylolpropane triacrylate (Sartomer SR-492), tripropyleneglycol diacrylate and mixtures thereof. These monomers are characterized by high reactivity/high double bond conversion, good mechanical properties, low polymerization shrinkage and relatively low viscosity.

Further suitable monomers are acrylamides such as N-ethyl acrylamide, N,N-dimethylacryl-amide, N-(2-hydroxyethyl)acrylamide, N,N′-diethyl-1,3-bis(acrylamido)-propane and 1,4-bis(acrylamido)butane. Compared to the monoacrylamides, the bisacrylamides are preferably used in excess in the organic binder.

The properties of the slurry before and after light curing can be influenced by a specific combination of monomers. Mixtures of monofunctional and difunctional monomers are characterized by a relatively low viscosity and reactivity of the resin mixture, whereby viscosity and reactivity decrease with the content of monofunctional monomers. A content of monofunctional monomers ensures lower brittleness and faster debinding of the green bodies obtained by light-curing the slurries. Mixtures of difunctional and trifunctional monomers have a higher reactivity, whereby the reactivity increases with the content of trifunctional monomers. The content of trifunctional monomers causes a higher brittleness and slower debinding of the green bodies.

Reactivity and viscosity of the resin mixture as well as the polymerization shrinkage are also determined by the molar mass of the monomers, whereby the polymerization shrinkage decreases with increasing molar mass, while the viscosity increases. Finally, the interaction with the material of the stereolithographic tank, e.g. the swelling of the material of the polymerization tank, can be influenced by the polarity of the monomers.

Preferred photoinitiators (b) for initiating radical photopolymerization are benzophenone, benzoin and their derivatives or diketones or their derivatives, such as 9,10-phenanthrenquinone, 1-phenylpropane-1,2-dione, diacetyl or 4,4′-dichlorobenzil. Particularly preferred are camphorquinone (CQ) and 2,2-dimethoxy-2-phenyl-acetophenone, and especially preferred are α-diketones in combination with amines as reducing agents, such as 4-(dimethylamino)-benzoic acid ester (EDMAB), N,N-dimethyl aminoethyl methacrylate, N,N-dimethyl-sym.-xylidine or triethanolamine.

Particularly preferred photoinitiators are Norrish type I photoinitiators, especially monoacyl or bisacyl phosphine oxides, and especially monoacyltrialkyl or diacyldialkyl germanium compounds, such as benzoyl trimethyl germanium, dibenzoyl diethyl germanium or bis(4-methoxy benzoyl)diethyl germanium (MBDEGe). Advantageously, mixtures of the various photoinitiators can also be used, such as bis(4-methoxy benzoyl) diethyl germanium in combination with camphorquinone and 4-dimethylaminobenzoic acid ethyl ester.

Very particularly preferred are camphorquinone (CAS No. 10373-78-1) in combination with ethyl 4-(dimethylamino)benzoate (EMBO, CAS No. 10287-53-3), as well as Norrish type I photoinitiators, in particular 2,4,6-trimethylbenzoyldipenylphosphine oxide (TPO, CAS No. 75980-60-8), ethyl(2,4,6-trimethyl benzoyl) phenyl phosphinate (TPO-L, CAS No. 84434-11-7), phenyl-bis(2,4,6-trimethylbenzoyl)phosphineoxide (Irgacure 819, CAS 162881-26-7), bis(2,6-difluoro-3-(1-hydropyrrol-1-yl)phenyl)titanocene (Irgacure 784, CAS no. 125051-32-3), 2-benzyl-2-(dimethylamino)-4′-morpholinobutyrophenone (Irgacure 369, CAS no. 119313-12-1), 1-butanone-2-(dimethylamino)-2-(4-methylphenyl)methyl-1-4-(4-morpholinyl) phenyl (Irgacure 379, CAS No. 119344-86-4) and especially bis(4-methoxybenzoyl)diethyl germanium (MBDEGe; Ivocerin).

As component (c), the slurries according to the invention contain ceramic and/or glass particles and/or glass-ceramic particles. Ceramics are understood to be inorganic materials that have a crystalline structure and are usually produced from corresponding powders. Preferably, the ceramics are produced by sintering (sintered ceramics). Oxide ceramics are preferably obtained by sintering metal oxide powders such as ZrO₂ or Al₂O₃.

According to the invention, slurries containing glass and/or glass ceramic particles as component (c) are preferred. Glass-ceramics are materials which are usually produced from amorphous glasses, in particular silicate glasses, by controlled crystallization and in which a glass phase and one or more crystal phases are present side by side in the solid. Glass or glass-ceramic particles comprising leucite, apatite and, most preferably, lithium disilicate crystals are particularly preferred. Glass-ceramics can be produced from appropriately composed glass powders that do not contain any crystallites by thermal treatment (crystallization and sinter firing). According to one embodiment, the slurries according to the invention can therefore contain glass powders which are not converted into the corresponding glass ceramic until the dental restoration is sintered.

According to a further preferred embodiment, mixtures of glass and glass-ceramic powder can be used in addition to pure glass or glass-ceramic powder. Preferred mixtures contain 0.5 to 99.99% by weight of glass-ceramic powder, based on the total mass of glass and glass-ceramic powder, with the proportion of glass powder preferably corresponding to the difference to 100% by weight. In this way, the sintering activity and dimensional stability can be adjusted. Glasses have a higher sintering activity (fewer pores, smoother surface), while glass-ceramic powders have a higher dimensional stability (no distortion). In addition, glass-ceramic powders in the slurry are chemically more stable against the dissolution of ions and the formation of carbonates and other undesirable products, resulting in more stable slurry properties.

Preferred glass-ceramics according to the invention are described in detail in EP 1 505 041 A1, and corresponding U.S. Pat. No. 8,042,358 B2, which US patent is hereby incorporated by reference, EP 2 261 184 A1, EP 2 377 830 A1 and corresponding U.S. Pat. No. 8,536,078 B2, which is hereby incorporated by reference, and EP 2 377 831 A1 and corresponding U.S. Pat. No. 8,536,078 B2, which US patent is hereby incorporated by reference. Particularly preferred are the lithium silicate materials described in DE 103 36 913 A1 and corresponding U.S. Pat. No. 8,047,021 B2, which US patent is hereby incorporated by reference, and particularly preferred are the lithium disilicate glass-ceramics described in EP 0 827 941 A1.

The ceramic, glass or glass-ceramic particles used as component (c) are preferably colored, and it is particularly preferred to use differently colored slurries to produce the core and shell structure. In order to achieve a natural appearance of the dental restoration, which imitates the core-shell impression of the tooth caused by dentin and enamel, the slurry for producing the core is colored darker and more opaque and the slurry for producing the shell structure is colored lighter and more transparent to match the natural enamel.

For this purpose, the ceramic, glass and/or glass-ceramic particles are preferably mixed with one or more pigments. Inorganic pigments, such as the oxides of Pr, Tb, Ce, Co, Ni, Cu, Bi, La, Nd, Sm, Eu, Gd and in particular Fe, Mn, Cr, Er, are particularly preferred. Colored spinel compounds of the type AB₂O₄, where A is preferably an alkali or alkaline earth metal ion and B is a transition metal ion of a higher oxidation state than A, can also be advantageously used to color the ceramic or glass-ceramic powders. The spinels are particularly suitable for coloring glass-ceramics and especially lithium disilicate glass-ceramics. The coloring components are added in the amount required to achieve the desired coloration. Preferably, the type and quantity of the coloring component(s) are selected in such a way that tooth-colored ceramic moldings are obtained after debinding and sintering. The color impression is only finally formed during sintering. Typically, the coloring components are each used in an amount of 0.01 to 1 wt. %, preferably 0.05 to 0.5 wt. % based on the total mass of component (c). Alternatively, the ceramic, glass or glass-ceramic powders can also be colored in accordance with EP 0 827 941 A1 by the addition of glass-coloring oxides (so-called ion coloring). Preferred glass-coloring oxides are TiO₂, CeO₂ and/or Fe₂O₃.

To achieve the desired color impression, it is also possible to use mixtures of differently colored ceramic, glass or glass-ceramic powders.

The particle size of component (c) is preferably in the range of 10 nm to 100 μm, preferably 100 nm to 10 μm. It depends on the ceramic used. For Al₂O₃, the size of the particles used as component (c) is preferably in the range from 50 to 500 nm, particularly preferably between 75 and 200 nm; for glass powder and/or glass ceramic powders in the range from 500 nm to 50 μm, most preferably between 1 and 15 μm; for TZP-3Y zirconium dioxide in the range from 50 to 500 nm, most preferably between 50 and 350 nm. The particle size is preferably selected in such a way that sedimentation-stable slurries are obtained. The specified lower and upper limits of the particle size ranges are the D10 and D90 values respectively. A range of 500 nm to 50 μm therefore means that the D10 value is 500 nm and the D90 value is 50 μm, i.e. 10% by volume of the particles are smaller than 500 nm and 90% by volume of the particles are smaller than 50 μm.

Furthermore, ceramic, glass or glass ceramic particles with a particle size in the range from 10 to 200 nm can also be used as nano- or organosols, i.e. as a dispersion of the nanoparticles in a solvent, a suitable monomer of component (a) or a mixture thereof. This is also the D10 or D90 value.

The particle size determination in the range of 0.1 μm to 1000 μm is preferably carried out using static light scattering (SLS), for example with a static laser scattering particle size analyzer LA-960 (Horiba, Japan) or with a Microtrac S100 particle size analyzer (Microtrac, USA). The light sources used are a laser diode with a wavelength of 655 nm and an LED with a wavelength of 405 nm. The use of two light sources with different wavelengths makes it possible to measure the entire particle size distribution of a sample in just one measurement run, whereby the measurement is carried out as a wet measurement. For this purpose, an aqueous dispersion of the filler is prepared and its scattered light is measured in a flow cell. The scattered light analysis for the calculation of particle size and particle size distribution is carried out according to the Mie theory according to DIN/ISO 13320. The measurement of the particle size in a range from 1 nm to 0.1 μm is preferably carried out by dynamic light scattering (DLS) of aqueous particle dispersions, preferably with a He—Ne laser with a wavelength of 633 nm, at a scattering angle of 90° and at 25° C., e.g. with a Malvern Zetasizer Nano ZS (Malvern Instruments, Malvern UK).

In the case of aggregated and agglomerated particles, the primary particle size can be determined using TEM images. Transmission electron microscopy (TEM) is preferably performed with a Philips CM30 TEM at an acceleration voltage of 300 kV. For sample preparation, drops of the particle dispersion are applied to a 50 Å thick copper grid (mesh size 300 mesh) coated with carbon and the solvent is then evaporated. The particles are counted and the arithmetic mean is calculated.

Particularly preferred according to the invention are glasses and lithium disilicate glass-ceramics with the following composition:

| | |
|---|---|
| SiO₂ | 57.0 to 80.0% by weight |
| Al₂O₃ | 0 to 5.0% by weight |
| La₂O₃ | 0.1 to 6.0% by weight |
| MgO | 0 to 5.0% by weight, in particular |
| | 0.1 to 5.0% by weight |
| ZnO | 0 to 8.0% by weight |
| K₂O | 0 to 13.5% by weight |
| Li₂O | 11.0 to 19.0% by weight |
| P₂O₅ | 0 to 11.0% by weight |
| Color components | 0 to 8.0% by weight |
| Additional components | 0 to 6.0% by weight |

whereby

| | |
|---|---|
| Al₂O₃ + La₂O₃ is | 0.1 to 7.0% by weight and |
| MgO + ZnO is | 0.1 to 9.0% by weight |

and wherein the color components are formed from glass coloring oxides and/or color bodies in the following amounts:

| | |
|---|---|
| glass-coloring oxides | 0 to 5.0% by weight and |
| Color bodies | 0 to 5.0% by weight. |

Preferred glass-coloring oxides are TiO₂, CeO₂ and/or Fe₂O₃. Preferred color bodies are the metal oxides commonly used in dental glass-ceramics, such as the above-mentioned inorganic pigments, and in particular commercially available isochromic color bodies, such as colored or doped spinels and/or doped ZrO₂. The color bodies can be both non-fluorescent and fluorescent materials. Unless otherwise stated, all percentages herein refer to the total mass of the glass-ceramic.

Preferred quantity ranges exist for the individual components of the preferred glass or lithium disilicate glass-ceramic according to the invention. These can be selected independently of one another and are as follows:

| | |
|---|---|
| SiO₂ | 57.0 to 75.0% by weight |
| Al₂O₃ | 0 to 2.5% by weight |
| La₂O₃ | 0.1 to 4.0% by weight |
| MgO | 0.1 to 4.0% by weight |
| ZnO | 0 to 6.0% by weight, in particular |
| | 0.1 to 5.0% by weight |
| K₂O | 0 to 9.0% by weight, in particular |
| | 0.5 to 7.0% by weight |
| Li₂O | 13.0 to 19.0% by weight |
| P₂O₅ | 0 to 8.0% by weight, in particular |
| | 0.5 to 8.0% by weight |
| Color components | 0.05 to 6.0% by weight |
| Additional components | 0 to 3.0% by weight. |

In addition to the components mentioned, the glass or the lithium disilicate glass-ceramic can contain other additional components, in particular B₂O₃, F, Na₂O, ZrO₂, BaO and/or SrO. The viscosity of the residual glass phase of the glass ceramic can be influenced by B₂O₃ and F, and it is assumed that they shift the ratio of surface to volume crystallization in favor of surface crystallization.

Preferably, the glass or lithium disilicate glass-ceramic consists essentially of the aforementioned components.

To produce the glass or lithium disilicate glass-ceramic, a starting glass containing the above-mentioned components, with the exception of the color bodies, is melted at temperatures of 1200 to 1650° C. For this purpose, suitable starting materials, such as carbonates, oxides and fluorides, are intimately mixed together and heated to the specified temperatures. If coloring oxides are to be used, these are added to the mixture. The resulting molten glass is poured into water to form a glass granulate and the glass granulate is then crushed into a powder with the desired particle size. Any color bodies present are then added to the powder. This glass powder can then be used to produce the desired slurry. In this case, a glass ceramic is formed during the sintering process. Preferably, the glass powder is subjected to a heat treatment in the temperature range from 400 to 1100° C. The heat treatment serves to trigger the crystallization of the initial glass and thus to form the glass ceramic, which is present after the heat treatment is complete and which can then be used to produce a slip. As mentioned above, mixtures of glass and glass-ceramic powder can also be used to produce the slips.

The glasses and lithium disilicate glass-ceramics preferred according to the invention and their production are described in detail in EP 0 827 941 A1, whereby the glass-ceramics specifically described there are particularly preferred.

According to a preferred embodiment of the invention, the particles of component (c) are surface-modified with suitable substances. For surface modification, compounds are preferably used which are chemically bound, i.e. by ionic or covalent bonds, to the surface of the ceramic, glass and/or glass-ceramic particles. Preferred are compounds that contain either acid groups, preferably carboxylic acid, phosphonic acid, hydrogen phosphate groups or acidic phosphorus acid ester groups, or silyl groups, preferably alkoxysilyl groups. The particle surface can be partially or preferably completely covered with the modifier. The modifying agents used according to the invention are monomeric compounds.

According to the invention, such compounds are particularly suitable which, in contrast to the so-called adhesion promoters or coupling reagents, only contain groups which react with the particle surface, but no radical polymerizable groups which form a covalent bond with the resin matrix (a). Such compounds are referred to herein as non-polymerizable surface modifiers. These compounds have the advantage that a stable bond between the particle surface and the polymer matrix does not occur in the cured green body, which simplifies the complete removal of the polymer components in the debinding process.

Suitable non-polymerizable surface modifiers are in particular linear or branched carboxylic acids, such as formic acid, acetic acid, propionic acid, octanoic acid, isobutyric acid, isovaleric acid, pivalic acid or phosphonic acids, e.g. such as methyl, ethyl, propyl, butyl, hexyl, octyl or phenylphosphonic acid. Silanes suitable as non-polymerizable surface modifiers are, for example, propyltrimethoxysilane, phenyltrimethoxysilane, hexyltrimethoxysilane, octyl-trimethoxysilane, trimethylchlorosilane, trimethylbromosilane, trimethylmethoxysilane or hexamethyldisilazane. Particularly preferred are acidic phosphoric acid esters, such as dimethyl, diethyl, dipropyl, dibutyl, dipentyl, dihexyl, dioctyl or di(2-ethylhexyl) phosphate.

Polymers, in particular polyelectrolytes, e.g. polycarboxylic acids or polycarboxylic acid salts, or non-ionic polymers, such as polyethylene glycol or carboxymethyl cellulose, are also suitable as surface modifiers for aqueous slurries. Polyelectrolytes that carry ionic groups, such as ammonium polycarboxylate, can adsorb relatively easily to the surface of solids and thereby impart an electrical charge to the particles. In the case of organic, non-aqueous slurries, polymers that are soluble in the polyreaction resin are suitable. Modifiers with a molecular weight in the range of 50 to 5,000 g/mol are preferred.

Surface modifiers are optionally used in an amount of preferably 0.1 to 5% by weight, particularly preferably 0.2 to 2% by weight and most preferably 0.5 to 1.5% by weight based on the mass of the slurry.

The slurries according to the invention may contain one or more non-ionic surfactants as component (d). Non-ionic surfactants are substances with surface-active properties that do not form ions in aqueous media. They are molecules that have a hydrophobic and a hydrophilic part. The overall hydrophobicity of the molecules can be adjusted by selecting the length and type of the hydrophobic and hydrophilic parts.

According to the invention, non-ionic surfactants with an HLB value in the range from 3 to 16, in particular from 4 to 13 and especially from 4 to 10 are preferred. The HLB value is determined according to the Griffin method.

Preferred non-ionic surfactants (d) are the ethoxylates of fatty alcohols, oxoalcohols or fatty acids, fatty acid esters of sugars and hydrogenated sugars, alkyl glycosides and block polymers of ethylene and propylene oxide, in particular short-chain block co-oligomers.

Particularly preferred are fatty acid esters of hydrogenated sugars, especially those with the formula R′—CO—O-sugar, where R′ is a branched or preferably straight alkyl radical with 10 to 25 carbon atoms, preferably 12 to 22 carbon atoms. Straight alkyl radicals with 16 to 22 carbon atoms are particularly preferred. “Sugar” stands for a hydrogenated sugar residue, which is preferably ethoxylated 1 to 5 times. Particularly preferred are fatty acid esters of sorbitol, especially sorbitan stearates such as sorbitan monostearate (CAS 1338-41-6).

A further particularly preferred group of surfactants are ethoxylates of fatty acids, in particular those with the general formula R″—(CO)—(OCH₂CH₂)ₘ—OH, in which R″ is a branched or preferably straight alkyl radical with 10 to 25 carbon atoms, preferably 12 to 22 carbon atoms. Straight alkyl radicals with 16 to 22 carbon atoms are particularly preferred. m is an integer from 2 to 20, preferably 2 to 10 and particularly preferably 2 to 6.

Particularly preferred surfactants (d) according to the invention are ethoxylates of fatty alcohols, in particular polyalkylene glycol ethers having the general formula R—(OCH₂CH₂)ₙ—OH, in which R is an alkyl radical having 10 to 20 carbon atoms and n is an integer from 2 to 25. R can be a branched or preferably straight alkyl radical, with alkyl radicals having 12 to 22 carbon atoms and in particular straight alkyl radicals having 12 to 22 carbon atoms being preferred. Particularly preferred alkyl radicals are lauryl, cetyl, cetearyl and stearyl.

The polyalkylene glycol ethers are obtained by reacting the corresponding fatty alcohols with ethylene oxide (EO). The index n indicates the number of ethylene oxide residues. Polyalkylene glycol ethers with 2 to 21 (n=2-21), in particular 2 to 12 (n=2-12) and especially 2 to 5 (n=2-5) ethylene oxide radicals are preferred.

Examples of preferred polyalkylene glycol ethers according to the invention are compounds in which R is a cetyl radical (C₁₆ radical) and n is 20 and in particular 2. These compounds have the INCI designations Ceteth-2 and Ceteth-20. Ceteth-2, for example, has the formula C₁₆H₃₃—(OCH₂CH₂)₂—OH.

Further preferred are compounds in which R is a stearyl radical (Ca radical) and n is 2, 10, 20 or 21. These compounds have the INCI designations steareth-2, steareth-10, steareth-20 and steareth-21. Steareth-2, for example, has the formula C₁₈H₃₇—(OCH₂CH₂)—OH.

Particularly preferred non-ionic surfactants are steareth-20 (HLB=15.3), steareth-10 (HLB=12.4), ceteth-20 (HLB=12.9) and especially steareth-2 (HLB=4.9) and ceteth-2 (HLB=5.3).

Mixtures of different non-ionic surfactants and in particular different polyalkylene glycol ethers can also be used.

INCI stands for International Nomenclature of Cosmetic Ingredients. It is an international guideline for the correct declaration of ingredients in cosmetics. According to the invention, it was surprisingly found that the use of non-ionic surfactants, which are normally used in the manufacture of cosmetic and pharmaceutical products, significantly improves the properties of slurries for the stereolithographic production of ceramic and glass-ceramic bodies.

It was found that the non-ionic surfactants used according to the invention are homogeneously miscible with radically polymerizable monomers and in particular with acrylates and methacrylates and do not impair the polymerization of these monomers. In the stereolithographic processing of the slurries according to the invention in the liquid state, a high reactivity and short exposure and processing times can thus be ensured. Advantageously, the non-ionic surfactants are also readily miscible with short-chain polar and highly reactive monomers.

The non-ionic surfactants according to the invention are preferably solid at a temperature of <20° C. and particularly preferably <30° C. As a result, they lead to solidification of the slurries, i.e. the slurries have a paste-like to solid consistency at a temperature of <20° C. and preferably <30° C. One advantage of this is that the homogeneous solidification prevents sedimentation of the particles and thus significantly increases the storage stability when stored at room temperature in the solid state. In addition, the non-ionic surfactants improve the strength of the polymerized objects (green bodies) at room temperature compared to green bodies made from slurries without the component (d).

Preferred are slurries which contain a non-ionic surfactant with a melting point of 20° C. to 120° C., preferably 20° C. to 100° C., particularly preferably 20° C. to 60° C. The slurry components are homogeneously miscible above the melting point of component (d) and solidify homogeneously below the melting point of (d). This effectively prevents sedimentation of the particles below the melting point of component (d). This ensures the stability of the mixture over a long period of time.

It is particularly advantageous that the non-ionic surfactants used according to the invention effectively prevent premature sedimentation of the particles even in the liquid state, resulting in a high stability of the slurry during processing.

Due to the good dispersing properties of the non-ionic surfactants for particles, the slurries can also be filled to a significantly higher degree with ceramic, glass and/or glass-ceramic powder, which results in a higher green density.

A further advantage of the non-ionic surfactants (d) preferred according to the invention is that they melt during debinding and flow or evaporate out of the matrix due to their relatively low molecular weight. They do not hinder the debinding process, but rather create channels that favor the release of the decomposition products of the organic matrix and thus facilitate debinding. During the heating process in the oven, the molecules of the non-ionic surfactant (d) located between the polymer chains of the matrix flow out of the green body in liquid form before the polymerized parts of the mixture are depolymerized and then pyrolyzed. The risk of defects such as cracks or even the destruction of the molded body during the debinding process is thus at least greatly reduced and usually completely eliminated. In addition, the flowing out/evaporation of component (d) already removes part of the organic mass at a comparatively low temperature, and the further heating process until all organic components have been completely removed can be carried out quickly, whereas previously only relatively low heating rates were possible.

In addition to the above-mentioned components, the slurries according to the invention preferably additionally contain at least one additive selected from colorants, UV absorbers, optical brighteners, solvents, inhibitors, debinding accelerators, defoaming agents and/or skin formation inhibitors.

Organic dyes are preferred as colorants, in particular azo dyes, carbonyl dyes, cyanine dyes, azo methines and methines, phthalocyanines and dioxazines. Particularly preferred are dyes that are soluble in the slurry, especially azo dyes. A particularly preferred dye is (4-(4-nitro phenyl azo) aniline (Disperse Orange 3, CAS No. 730-40-5). Furthermore, dyes which absorb in the same wavelength range as the polymerization initiator are particularly preferred. Particularly preferred are dyes that have an absorption maximum that corresponds to the wavelength of the light used for curing. Dyes with an absorption maximum in the range from 350 to 550 nm, preferably 380 to 480 nm, are very advantageous.

In contrast to the coloring components used to color the ceramic or glass-ceramic powder, colorants are understood here to be substances that burn completely during debinding and sintering. These colorants are used solely to color the slurry and thus facilitate its processing. They are not used to color the ceramic. Coloring can be helpful in making it easier to differentiate between slips. In addition, dyes absorb the penetrating light during stereolithographic printing and reduce its penetration depth into the material, making it possible to print objects more precisely, which is particularly advantageous with very transparent slips.

Preferred UV absorbers are 2,2′-methylenebis[6-(2H-benzotriazol-2-yl)-4-(1,1,3,3-tetramethylbutyl)phenol], 2,2′,4,4′-tetrahydroxybenzophenones, 2-tert-butyl-6-(5-chloro-2H-benzotriazol-2-yl)-4-methylphenol, 2,2′-benzene-1,4-diylbis(4h-3,1-benzoxazin-4-one, 2-(4,6-bis(2,4-dimethylphenyl)-1,3,5-triazin-2-yl)-5-(octyloxy)-phenol, 2-(2-hydroxy-5-methylphenyl)-benzo triazoles and 2-(2H-benzotriazol-2-yl)-6-dodecyl-4-methylphenol. A preferred optical brightener according to the invention is 2,5-bis(5-tert-butyl-benzoxazol-2-yl)thiophene. Particularly preferred are UV absorbers and optical brighteners which have an absorption maximum which corresponds to the wavelength of the light used for curing.

Organic solvents are preferred as solvents, in particular solvents which have a boiling temperature of at least approx. 120° C., preferably from 150 to 250° C., so that premature evaporation does not occur during stereolithographic processing of the slurry. Mixtures of solvents that can be gradually vaporized in a temperature range between 15° and 250° C. are particularly suitable. Particularly suitable are n-octanol, triethylene glycol divinyl ether, 2-amino-2-methyl-1-propanol, 2-methyl-2,4-pentanediol, tripropylene glycol, tetraethylene glycol, triethylene glycol, triethyl citrate, ethyl acetoacetate, cyclohexanol, cyclohexanone, diethylene glycol monomethyl ether, oxalic acid dibutyl ester, 2,5-dimethoxy tetrahydrofuran, polyethylene glycol 300, 1- or 2-nonaneol, diethylene glycol diethyl ether and mixtures thereof.

Preferred solvents are also polyethylene glycols (PEG), polypropylene glycols (PPG), PEG-PPG co-polymers, glycerol and glycerol derivatives, in particular ethoxylated and/or propoxylated glycerol, as well as phthalates, benzoates and cycloaliphatic esters. Particularly preferred are solvents with an Mw of less than 5,000 g/mol, preferably less than 1,000 g/mol. Suitable solvents are compounds which are liquid at room temperature and which do not react with the other components of the slurry under storage and application conditions. Particularly preferred solvents are PEG 200-600 g/mol, PPG 200-800 g/mol, Co-PEG-PPG 200-800 g/mol and especially polypropylene glycol ˜400 g/mol, diethyl phthalate, dimethyl phthalate, ethyl benzoate, butyl benzoate, benzyl benzoate, diethylene glycol dibenzoate, 1,2-cyclohexanedicarboxylic acid diisononyl ester. Advantageously, mixtures of these solvents can also be used.

The solvent or solvents are preferably used in a total amount of 0 to 50% by weight, particularly preferably 3 to 20% by weight, based on the mass of the slurry.

It was found that the evaporation of the above solvents additionally promotes the formation of micropores in the green body. These pores close in the same way as the channels formed by the outflow or evaporation of the non-ionic surfactants during sintering. They facilitate the escape of gases during debinding and thus prevent the formation of stresses and cracks. In addition, the risk of separation of the stereolithographically produced layers is reduced and complete removal of all organic components is facilitated.

Alternatively, the porosity of the green body can also be increased by extractive removal of leachable components prior to heat treatment. Suitable extractable components must be matched to the extraction agent. Water can be used as an extraction agent for oxide ceramic particles. In this case, water-soluble polymers such as polyvinyl alcohol, polyvinylpyrrolidone and polyethylene glycols are suitable. For ceramic, glass and glass-ceramic particles that are sensitive to water, organic solvents (especially apolar solvents) can be used for extraction. In this case, for example, petrol-soluble substances such as long-chain fatty acid esters are suitable as extractable components. The preferred amount of extractable components is between 0 and 30% by weight, particularly preferably between 1 and 20% by weight, based on the mass of the slurry.

The slurries according to the invention can advantageously contain an inhibitor as a stabilizer as a further additive to prevent a spontaneous polyreaction. The inhibitors or stabilizers improve the storage stability of the slurries and also prevent an uncontrolled polyreaction in the stereolithographic tank. The inhibitors are preferably added in such an amount that the slurries are stable in storage for a period of approx. 2 to 3 years. Particularly preferably, the inhibitors are used in an amount of 0.001 to 1.0% by weight, most preferably 0.001 to 0.50% by weight, in each case based on the total mass of the slurry.

Preferred are so-called aerobic inhibitors, in particular phenols, such as hydroquinone monomethyl ether (MEHQ) or 2,6-di-tert-butyl-4-methyl-phenol (BHT), which are only effective in the presence of oxygen and are preferably used in a concentration range of 100 to 2000 ppmw. Suitable anaerobic inhibitors are phenothiazine, 2,2,6,6-tetramethyl-piperindine-1-oxyl radical (TEMPO), iodine and copper (I) iodide. These are already effective in low concentrations of preferably 10 to 50 ppm, even in the absence of oxygen. Polymerization only takes place when these additives have been used up. It is advantageous to use a mixture of aerobic and anaerobic inhibitors.

Aerobic inhibitors are preferably used in an amount of 0.001 to 0.50% by weight and anaerobic inhibitors in an amount of 0.001 to 0.02% by weight, in each case based on the total mass of the slurry. Preferred mixtures contain 0.005 to 0.10% by weight of aerobic inhibitors and 0.001 to 0.01% by weight of anaerobic inhibitors, also based on the total mass of the slurry.

According to a further embodiment of the invention, the slurries contain a so-called debinding accelerator as an additional additive. This is preferably used in an amount of 0 to 20% by weight, particularly preferably 0.01 to 10% by weight, in each case based on the total mass of the slurry. Debinding accelerators are substances that facilitate the removal of the binder during the debinding process.

The debinding of the green body can, for example, be promoted or specifically influenced by poly-reaction-active substances in the polyreactive resin. On the one hand, these are additives that influence network formation, such as chain transfer-active substances, so-called chain regulators, which lead to a reduction in the polymer network density and thus to better thermal degradability. Known chain regulators, e.g. for the radical polymerization, are primarily mercaptans, such as lauryl mercaptan, and disulfides. Disulphides, especially dithiourethane disulphides, such as tetramethylthiuram disulphide or isopropylxanthogenic acid disulphide, act as so-called photoiniferters during radical photopolymerization. These are compounds that act both as photoinitiators (photoini-) and participate in transfer reactions (-fer-) and termination reactions (-ter) (see T. Ostsu, M. Yoshida, Makromol. Chem., Rapid. Commun. 3 (1982) 127-132: Role of Initiator-Transfer Agent-Terminator (Iniferter) in Radical Polymerizations: Polymer Design by Organic Disulfides as Iniferters). The addition of chain-transfer active substances, i.e. chain regulators or photoiniferters, causes a reduction in the network density of the polyreaction network, with almost unchanged reactivity of the polyreaction resin mixture. Chain regulators and photoiniferters are preferably used in an amount of 0.005 to 25 wt. % each and particularly preferably 0.01 to 10 wt. % relative to component (a).

Comonomers that lead to a reduction in the thermal stability of polymer networks can also be used as debinding accelerators. Comonomers containing thermally labile groups, such as peroxide, azo or urethane groups, which are incorporated into the polymer network during the stereolithographic process and then accelerate the degradation of the polymer network in the thermal debinding process, are suitable for this purpose. A preferred example of a polymerizable peroxide is 4,4′-divinylbenzoyl peroxide, which is accessible by reacting 4-vinylbenzoyl chloride with sodium peroxide. A preferred example of an azo compound capable of polymerization is the ester of 2-hydroxyethyl methacrylate and 4,4′-azobis-(4-cyano-valeric acid). Preferred thermally labile urethanes are accessible from diisocyanates, for example by reacting 2,2,4-trimethylhexamethylene diisocyanate (TMDI) or toluene diisocyanate (TDI) with hydroxypropyl acrylate (HPA) or 2-hydroxyethyl acrylate (HEA). Another example of a thermally labile monomer building block is α,α,α′,α′-tetramethyl-1,4-benzene dimethyl acrylate, whose incorporation into a Michael addition network, for example of diacrylates and diacetoacetates, leads to accelerated degradation of the polymer network in the presence of catalytic quantities of an acid.

Comonomers whose polyreaction products are easily thermally degradable are also suitable as debinding accelerators. Comonomers which, like α-methylstyrene, have a low ceiling temperature T_{c} are preferred for radical polymerization resins. The ceiling temperature is the limiting temperature at which the polymerization is in equilibrium with the depolymerization and can be calculated from the quotient of the polymerization enthalpy and the polymerization entropy (see H.-G. Elias, Makromolekule [Macromolecules], Vol. 1, 6^{th} ed., Wiley-VCH, Weinheim etc. 1999, 193 ff.). For example, T_{c} for α-methylstyrene is 61° C. The ceiling temperature T_{C} of polytetrahydrofuran (PTHF) is 80° C. Accordingly, the degradability of poly(meth)acrylate networks can be accelerated, for example, by using telechelic polymers, in particular PTHF di(meth)acrylate telechelic polymers, as a comonomer. According to the invention, comonomers with a ceiling temperature of −10 to 150° C., preferably 10 to 150° C. and particularly preferably 20 to 130° C. are particularly suitable. The comonomers are preferably used in an amount of 0.1 to 30% by weight and particularly preferably 0.5 to 20% by weight based on component (a).

Furthermore, the slurries according to the invention may contain additives that promote the oxidative degradation of the polymer matrix during the debinding process, such as peroxides that are stable at room temperature, or also catalytically active components that enable catalytic debinding. In addition to peroxides, other substances that have an oxidizing effect, such as nitric acid, or that split off or form oxidizing agents, are also suitable.

In addition, the slurries according to the invention may contain defoaming agents and/or skin-preventing agents which prevent the formation of foam during the production of the slurries or the formation of a skin during the processing of the slurries. Defoaming agents and/or skin formation inhibitors are preferably used in an amount of 0 to 5% by weight, particularly preferably 0.1 to 2% by weight in the organic matrix, based on the mass of component (a).

The rheological properties of the slurries according to the invention are preferably adjusted so that their viscosity is in the range of 200 to 200,000 mPa·s, particularly preferably 500 to 100,000 mPa·s. The viscosity is determined at the desired processing temperature of the slurry using a plate-plate viscometer at a shear rate of 20/s. The processing temperature is preferably in the range from 10 to 100° C., more preferably 15 to 60° C. and most preferably between 2° and 50° C.

The slurries according to the invention preferably have the following composition:
5 to 65% by weight, preferably 9 to 57% by weight, particularly preferably 10 to 40% by weight of monomer (a);
0.001 to 1.0% by weight, preferably 0.01 to 1.0% by weight, particularly preferably 0.05 to 1.0% by weight of photoinitiator (b);
33 to 90% by weight, preferably 40 to 88% by weight, particularly preferably 56 to 86% by weight of ceramic, glass and/or glass-ceramic particles (c);
optionally 1 to 30% by weight, preferably 2 to 15% by weight, particularly preferably 3 to 10% by weight of nonionic surfactant (d).

The ceramic, glass and/or glass-ceramic particles (c) are preferably colored in the manner described above, for example mixed with coloring pigments.

Furthermore, the slurries contain preferably:
0 to 0.2% by weight, preferably 0 to 0.05% by weight, particularly preferably 0 to 0.02% by weight of colorant(s) for coloring the slurry; and/or
0.1 to 5% by weight, preferably 0.2 to 2% by weight, particularly preferably 0.5 to 1.5% by weight of surface modifying agent; and/or
0 to 50% by weight, preferably 5 to 20% by weight, particularly preferably 5 to 10% by weight of solvent.

Unless otherwise stated, all figures refer to the total weight of the slurry. The coloring components used to color the ceramic, glass and/or glass-ceramic particles are contained in the quantities stated for component (c).

According to the invention, slurries of the same category are used to produce the core structure and shell structure, i.e. glass, glass-ceramic or ceramic slurries, whereby the slurries can be colored differently. According to a particularly preferred embodiment of the invention, glass-ceramic and especially preferred lithium disilicate glass-ceramic slurries are used to produce the core and shell structure, which differ only in color and opacity after sintering and otherwise have the same composition. In this way, a particularly good match is achieved between the thermal expansion coefficients and the sintering shrinkage of the slips. If aesthetic aspects are less important, the core and shell structure can also be produced from the same slip.

It may be advantageous to use a slurry to produce the shell structure that contains a minimally lower or higher content of ceramic, glass and/or glass-ceramic particles (c) than the slurry used to produce the core structure. Preferably, the deviation is no more than ±1% by weight. This leads to a slightly greater sintering shrinkage of the shell structure. The resulting tension stabilizes the dental restoration. This is particularly the case with one-piece shell structures, which completely enclose the core and form a closed shell. Slightly different sintering shrinkage is particularly advantageous if a joining slip is to be used.

It may also be advantageous to produce the shell structure using a slurry containing ceramic, glass and/or glass-ceramic particles with a lower softening temperature (glass transition temperature) than the slurry used to produce the core. The difference in softening temperatures is preferably ≤50° C. This can improve the gloss of the dental restoration. In addition, if the softening temperatures of the core and shell material differ, sintering can be controlled in such a way that the porosity of the core is higher than that of the shell structure. In general, the aim of sintering is to achieve the highest possible density of the shell structure. If the density of the core is lower than that of the shell structure, it appears more opaque compared to the shell structure, which gives the restoration a more natural appearance.

A slurry with the composition defined above is also preferably used to join the green bodies of the core and shell structure. The color and transparency of this joining slip are preferably selected so that they lie between the color and translucency of the core and shell structure. If a joining slip is to be used, a joining gap is preferably provided at least in some aeras between the core and the shell structure, preferably with a width of 10 to 300 μm. The green bodies of the core and shell can be joined together, for example, by applying the joining slip to the green body of the core and then placing the green body or bodies of the shell structure on the core green body under slight pressure. Any excess of the joining slip is then removed.

The use of joining slip and joining gap has the advantage that so-called fastening webs, support structures etc., which are formed when printing the molded parts, only need to be removed before joining to the extent that they exceed the dimensions of the joining gap. Smaller remnants and inaccuracies are compensated for by the joining gap and the joining slip.

The joining slips are primarily used to fix the parts after joining. They should be stable so that they do not run off during application and form a stable slurry layer, and have sufficient strength and adhesion after joining. During joining, the parts to be joined are brought together in a form-fit. Joining can be facilitated by a vibrating table, for example, if required. The fixation of the parts caused by the joining slip is usually sufficient for further processing of the green bodies, so that photo-hardening of the joining slip is not absolutely necessary but can be advantageous.

The slurries according to the invention are sedimentation-stable in the liquid state. They provide good green body strength and high dimensional stability, accuracy and precision after debinding, sintering and cleaning. The green bodies have a high green density and can be debinded without deformation, cracking or stress formation. During sintering, they result in high-strength ceramics that are suitable for dental purposes. During sintering, a density of >98% of the theoretical density can easily be achieved. The slips are particularly suitable for the production of ceramic or glass-ceramic compacts, especially for the production of dental restorations such as inlays, onlays, veneers, crowns, bridges or frameworks.

The green bodies are preferably produced using stereolithography. Stereolithographic printing is computer-aided on the basis of CAD data. The required data are obtained in the usual way by optical or mechanical digitization in the patient's mouth (intraoral) or on the basis of models (extraoral). The core and shell structure of the dental restoration are then designed on the computer on the basis of this data set. Tooth shapes stored in databases can be used for this. Two data sets are then created under software control, which are used to print the core and shell structure independently of each other. When designing the restoration, the material-related shrinkage behavior is taken into account accordingly.

The green bodies produced by the printing process already have a relatively high strength and can be handled without any problems. The green bodies of the core and shell structure are preferably cleaned and then assembled to form the green body of the dental restoration. As the sintering shrinkage and the coefficients of thermal expansion largely match, the green bodies can be produced with a precise fit so that they are held together in a form-fitting and/or force-fitting manner after joining, even without joining slip, and can be debinded and sintered. Preferably, however, a joining slip is used to join the components, especially if the shell structure does not completely enclose the core structure and/or is multi-part.

The stereolithographic production of the core and shell structure has the advantage that the moldings can be produced with great precision and do not show any milling marks, for example. Another advantage is that only one debinding and sintering process is required, which considerably reduces the time required to produce the dental restoration.

After sintering, the dental restorations can optionally be glazed and individually characterized with dental ceramic stains.

The method according to the invention and the materials used according to the invention are suitable for the production of dental restorations for the restoration of several teeth, such as bridges, and in particular for the production of dental restorations for the restoration of one tooth, such as inlays, onlays, veneers and, in particular, crowns. The multi-layer structure of the restorations enables improved esthetic properties to be achieved. However, by joining the core and shell structure in the green state and then sintering the components together, the multi-layer structure does not require any significant additional effort compared to the production of one-piece restorations. The preferred slurries according to the invention also result in a high green strength, which enables safe handling of the green bodies and ensures high stability during debinding. In addition, the green bodies can also be debinded very well after joining.

### EXAMPLES

### Example 1

### Fabrication of the Core and Shell Structure of an Anterior Crown

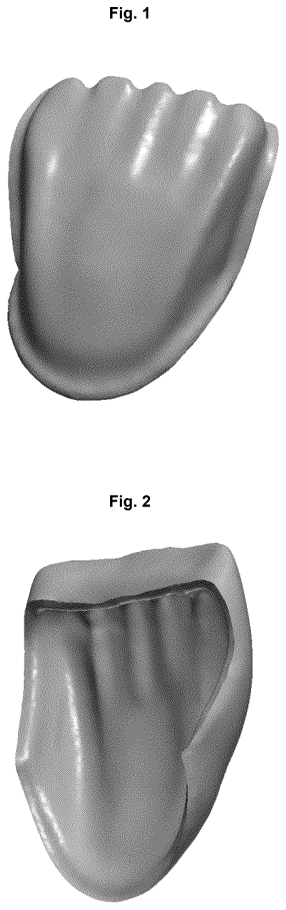

Using standard CAD software (EXOCAD v3.2; exocad GmbH, Rosa-Parks-Str. 2, D-64295 Darmstadt, Germany), a crown for an incisor was designed on the computer. The data set was then divided into two partial data sets. The first partial data set defines the core structure of the crown shown in FIG. 1 and the second partial data set defines the one-piece shell structure shown in FIG. 2. A joining gap with a width of 35 μm was provided between the core and shell structure. Both the core and shell structures have cusps that mimic natural mamelons. These structures facilitate the joining of the core and shell structure and give the dental restoration a natural appearance after joining.

The partial data sets were converted separately into processing data for a stereolithographic printing machine (Prograprint PR5; Ivoclar Vivadent AG) using standard CAM software (PrograPrint CAM software V1.2, Ivoclar Vivadent AG, Bendererstr. 2, 9494 Schaan, Liechtenstein). The printer was operated in a climate chamber at 40° C. to ensure the flowability of the slurry. The core structure and the shell structure were then printed separately under computer control. The slurries used for this had the following composition:

| | Shell | Core |
|---|---|---|
| Component | Slurry | Slurry |
| Tricyclodecanediol diacrylate (SR833S) | 9.12 | 9.12 |
| Bisphenol A dimethacrylate with 3 ethoxy groups | 12.125 | 11.61 |
| (SR-348c) | | |
| Photoinitiator (Ivocerin) | 0.23 | 0.23 |
| 4-(4-Nitrophenylazo)aniline 3 | 0.015 | 0.015 |
| (Disperse orange 3, CAS 730-40-5) | | |
| Rheology additive | 4.17 | 4.17 |
| (Solplus D540, CAS 1000871-74-8, Lubrizol) | | |
| Benzyl benzoate (solvent) | 4.5 | 4.5 |
| Diethylene glycol dibenzoate (solvent) | 0.34 | 0.34 |
| Lithium disilicate glass powder¹⁾ | 69.5 | 0 |
| (e. max Press, shade incisal S2, D50 = 6 μm)²⁾ | | |
| Lithium disilicate glass powder¹⁾ | 0 | 70.015 |
| (e. max Press, shade Dentin MT A3, D50 = 6 μm)² | | |
| Total | 100 | 100 |

¹⁾according to EP 0 827 941 A1
²⁾Particle size range: 500 nm to 50 μm

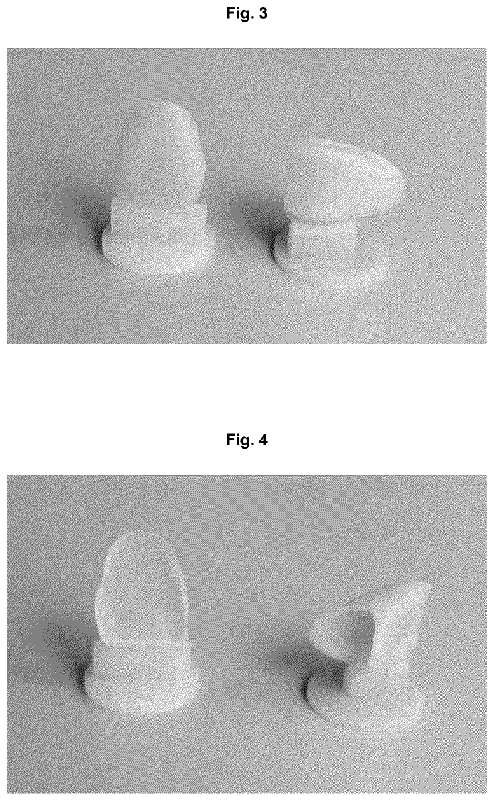

The printed green bodies were cleaned by rinsing with water and blown dry with compressed air. The resulting green bodies were then debinded by heating at a heating rate of 0.2 K/min to 500° C. for 30 minutes in a furnace (Nabertherm L9/11 BO; Lilienthal Bremen, Germany) and then sintered by heating at a heating rate of 10 K/min to 890° C. for 5 minutes in a sintering furnace (Programat P510; Ivoclar Vivadent AG; Schaan, Liechtenstein). The sintered structures are shown in FIGS. 3 and 4.

### Example 2

### Fabrication of an Anterior Crown

A crown for an anterior tooth was designed in the manner described in Example 1 and fabricated using the slips described in the following table.

| | Shell | Core | Joining |
|---|---|---|---|
| Component | Slurry | Slurry | slip |
| Tricyclodecanediol diacrylate (SR833S) | 9.12 | 9.12 | 9.12 |
| UDMA | 12.125 | 11.61 | 11.875 |
| Photoinitiator (Ivocerin) | 0.23 | 0.23 | 0.23 |
| 4-(4-Nitrophenylazo)aniline 3 | 0.015 | 0.015 | 0.015 |
| (Disperse orange 3, CAS 730-40-5) | | | |
| Rheology additive | 4.17 | 4.17 | 4.17 |
| (Solplus D540, CAS 1000871-74-8) | | | |
| Benzyl benzoate (solvent) | 4.5 | 4.5 | 4.5 |
| Diethylene glycol dibenzoate (solvent) | 0.34 | 0.34 | 0.34 |
| Lithium disilicate glass powder¹⁾ | 69.5 | 0 | 0 |
| (e. max Press, shade incisal S2, | | | |
| D50 = 6 μm)²⁾ | | | |
| Lithium disilicate glass powder¹⁾ | 0 | 70.015 | 0 |
| (e. max Press, shade Dentin MT A3, | | | |
| D50 = 6 μm)²⁾ | | | |
| Lithium disilicate glass powder¹⁾ | 0 | 0 | 69.75 |
| (e. max Press, shade incisal S2, | | | |
| D50 = 6 μm)²⁾ | | | |
| Total | 100 | 100 | 100 |

¹⁾according to EP 0 827 941 A1
²⁾Particle size range: 500 nm to 50 μm

The printed green bodies were cleaned by rinsing with water and blown dry with compressed air. The support structure of the cutting edge was removed using a rotating cutting disk (standard dental tool). The joining slip was then applied to the bonding surfaces of the core and shell, both molds were pressed together in a form-fitting manner and the excess bonding slurry was then removed.

The joined green bodies were subsequently light-cured with a dental light-curing unit (PrograPrint Cure, Program Wax, Ivoclar Vivadent AG) to increase the degree of polymerization. The bonding slurry was also cured.

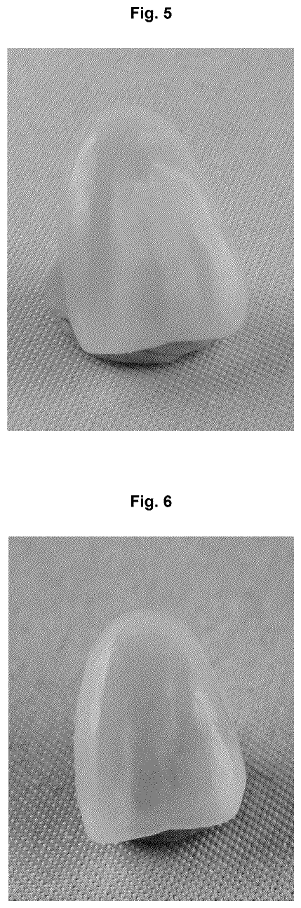

The green bodies obtained were then debinded by heating at a heating rate of 0.2 K/min to 500° C. for 30 minutes in a furnace (Nabertherm L9/11 BO, Lilienthal Bremen, Germany) and then sintered by heating at a heating rate of 10 K/min to 890° C. for 5 minutes in a sintering furnace (Programat P510; Ivoclar Vivadent AG; Schaan, Liechtenstein). The resulting crown is shown in FIG. 5. The crown shown in FIG. 5 was subsequently polished. The finished crown is shown in FIG. 6.

The slurry used to produce the shell structure was slightly less filled than the slurry used to produce the core. It therefore exhibited slightly greater sintering shrinkage than the core slurry, which led to an improvement in the bonding of the core and shell structure during sintering. The filling level of the joining slip was between the filling level of the core and shell slurry, resulting in a good transition between core and shell.

1. A process for the production of an all-ceramic dental restoration with a one-piece core structure and a one- or multi-part shell structure, comprising
constructing a digital model of the dental restoration to obtain a CAD data set,
dividing the CAD data set into at least two separate CAD partial data sets wherein a first partial data set defines a contour of the core structure and a second partial data set defines a contour of the shell structure,
fabricating a green body of the core structure of the restoration using the first partial data set comprising curing a first slurry by locally applying radiation energy to form a geometric shape of the core structure,
fabricating a green body of the shell structure of the restoration using the second partial data set comprising curing a second slurry by locally applying radiation energy to form a geometric shape of the shell structure,
combining the core and shell structures in the green state in order to obtain a green body of the dental restoration,
subjecting the green body of the dental restoration to a heat treatment to remove a binder to obtain a brown body of the dental restoration, and
sintering the brown body of the dental restoration to obtain the finished dental restoration.

2. The process according to claim 1, in which the CAD partial data set of the shell structure is divided into two or more CAD shell structure partial data sets, each comprising a part of the contour of the shell structure, the second slurry is cured by local introduction of radiation energy to form a geometric shape of a first component of the shell structure which is based on a first partial data set of the shell structure, separately, a further slurry is cured by local application of radiation energy to form a geometric shape of a second component of the shell structure which is based on a second partial data set of the shell structure, and this step is repeated according to a number of CAD shell structure partial data sets, and the core structure and the components of the shell structure are then joined together in the green state to obtain a green body of the dental restoration.

3. The process according to claim 2, in which the same slurry is used to produce the components of the shell structure, or a different slurry is used to produce each component of the shell structure.

4. The process according to claim 1, wherein slurries which differ in color and opacity but otherwise have the same composition are used to produce the core structure and to produce the shell structure.

5. The process according to claim 1, wherein the slurry used to produce the shell structure comprises a minimally lower or minimally higher content of ceramic, glass and/or glass-ceramic particles (c) than the slurry used to produce the core structure.

6. The process according to claim 1, in which the green bodies of the core structure and the shell structure are joined together using a joining slip and the joining slip is cured by polymerization after joining.

7. The process according to claim 1, wherein the green body of the dental restoration is debinded by removing the binder by heating the green body to a temperature of 90° C. to 600° C.

8. The process according to claim 1, wherein the brown body is sintered at a temperature of 650 to 1800° C.

9. The process according to claim 1, in which the slurry used to produce each of the green bodies of the core and shell structures comprises
(a) 5 to 65% by weight of at least one free-radically polymerizable monomer,
(b) 0.001 to 1.0% by weight of at least one photoinitiator and
(c) 33 to 90% by weight of ceramic particles and/or glass particles and/or glass-ceramic particles,
in each case relative to the total mass of the slurry.

10. The process according to claim 9, in which the slurry used to produce each of the green bodies comprises lithium disilicate glass-ceramic particles and/or glass particles for a lithium disilicate glass-ceramic.

11. The process according to claim 10, in which the slurry used to produce each of the green bodies comprises the glass or glass-ceramic particles having the following composition:

| | |
|---|---|
| SiO₂ | 57.0 to 80.0% by weight |
| Al₂O₃ | 0 to 5.0% by weight |
| La₂O₃ | 0.1 to 6.0% by weight |
| MgO | 0 to 5.0% by weight, in particular |
| | 0.1 to 5.0% by weight |
| ZnO | 0 to 8.0% by weight |
| K₂O | 0 to 13.5% by weight |
| Li₂O | 11.0 to 19.0% by weight |
| P₂O₅ | 0 to 11.0% by weight |
| Color components | 0 to 8.0% by weight |
| Additional components | 0 to 6.0% by weight |

whereby

| | |
|---|---|
| Al₂O₃ + La₂O₃ is | 0.1 to 7.0% by weight and |
| MgO + ZnO is | 0.1 to 9.0% by weight |

and wherein the color components are formed from glass coloring oxides and/or color bodies in the following amounts:

| | |
|---|---|
| glass-coloring oxides | 0 to 5.0% by weight and |
| color bodies | 0 to 5.0% by weight. |

12. The process according to claim 11, wherein the glass coloring oxide(s) is/are selected from TiO₂, CeO₂ and/or Fe₂O₃ and/or the color body/color bodies is/are selected from doped spinels and/or doped ZrO₂ and/or the additional component(s) is/are selected from B₂O₃, F, Na₂O, ZrO₂, BaO and/or SrO.

13. The process according to claim 9, wherein the slurry comprises a mixture of glass and glass-ceramic powder.

14. The process according to claim 9, wherein the free-radically polymerizable monomer (a) comprises an aliphatic urethane diacrylate, phthalic acid HEA ester, pyromellitic acid diHEA ester, bisphenol A di(meth)acrylate, bis-G(M)A (an addition product of (meth)acrylic acid and bisphenol A diglycidyl ether), an ethoxylated or propoxylated bisphenol A di(meth)acrylate, UD(M)A, triethylene glycol di(meth)acrylate (TEGD(M)A), tricyclodecane dimethanol di(meth)acrylate, ethoxylated or propopoxylated trimethylolpropane tri(meth) acrylate, tripropylene glycol diacrylate or a mixture thereof.

15. The process according to claim 9, wherein the photoinitiator (b) comprises camphorquinone (CAS No. 10373-78-1) in combination with ethyl 4-(dimethylamino)benzoate (CAS No. 10287-53-3), 2,4,6-trimethylbenzoyldiphenylphosphine oxide (CAS No. 75980-60-8), ethyl (2,4,6-trimethylbenzoyl)phenylphosphinate (CAS No. 84434-11-7), phenylbis(2,4,6-trimethylbenzoyl)phosphine oxide (CAS No. 162881-26-7), bis(2,6-difluoro-3-(1-hydropyrrol-1-yl)phenyl)titanocene (CAS No. 125051-32-3), 2-benzyl-2-(dimethylamino)-4′-morpholinobutyrophenone (CAS No. 119313-12-1), 1-butanone-2-(dimethylamino)-2-(4-methylphenyl)methyl-1-4-(4-morpholinyl)phenyl (CAS No. 119344-86-4), bis(4-methoxybenzoyl)diethylgermanium or a mixture thereof.
